(19) United States
(12) Patent Application Publication
(10) Pub. No.: US 2023/0130318 A1
(43) Pub. Date: Apr. 27, 2023

(54) METHOD AND APPARATUS FOR DETERMINING A MEASURE OF CONTACT OF EMG SENSORS

(71) Applicant: Emteq Limited, Brighton (GB)

(72) Inventors: Charles Nduka, Brighton (GB); Mahyar Hamedi, Brighton (GB); Andrew Cleal, Brighton (GB); Mohsen Fatoorechi, Brighton (GB)

(73) Assignee: Emteq Limited, Brighton, EN (GB)

(21) Appl. No.: 18/048,663

(22) Filed: Oct. 21, 2022

(30) Foreign Application Priority Data
Oct. 22, 2021 (GB) 2115241.8

(51) Int. Cl. A61B 5/395; A61B 5/00

(52) U.S. Cl. CPC A61B 5/395; A61B 5/7221; A61B 5/7257; A61B 5/7264

(57) ABSTRACT

A method of determining a measure of contact of an EMG sensor with the skin of a human or animal subject, the method comprising: receiving data captured by the EMG sensor; calculating a spectral entropy of the received data over a first time period in respect of a predetermined frequency band; determining a measure of contact for the first time period in dependence on the spectral entropy of the received data; and processing the received data of the first time period in dependence on the measure of contact.

### TECHNICAL FIELD

This invention relates to apparatus and methods for determining a measure of contact of EMG sensors.

### BACKGROUND

Traditionally, biological electrical activity sensors have been fixed to the body. For example, surface electromyography (EMG) recording sensors or electrocardiography (ECG) electrodes are commonly held against the body with adhesive tape or a self-adhesive sticker/pad.

As the quality of the signals obtained by such sensors is strongly dependent on the contact between the sensor and the body, care is taken to prepare the surface of the body to ensure a sound contact by removing hair and cleaning the surface with alcohol.

However, the use of such sensors is wholly unsuitable for certain applications. For example, on the face, or parts of the body which frequently move or where the skin frequently wrinkles and stretches can render the adhesives holding the sensors ineffective, and some users can develop allergies to the adhesives used. Furthermore, the onerous application process described above can render the sensors unsuitable for casual use, or use in the home by non-medical consumers.

To overcome some of these issues, systems using non-adhesive sensors have been developed in recent years. These systems, however, have encountered further problems related to sensors lifting from the surface of the body and artefacts in the signal caused by movement of the body, each of which degrades the quality of the measured signals and can lead to the capture of erroneous data.

A basic solution to the problem of sensor lift is to inject a fixed DC signal into the measurement circuit. When sensor lift occurs the measured signal voltage will rise and lift may be determined by simply ignoring all signals that are above some predefined level. However, when skin contact is made the voltage is discharged through the user within safe tolerances causing the measured signal to drop. However, such a solution is prone to false positives, i.e. some amount of useful data will be discarded. The solution is also only able to detect sensor lift a sometime after the event as many factors affect the rise and fall time of the measured signal.

Another common solution includes injecting a reference signal into the body at a frequency different from the biological signal of interest. For example, frequencies within the range of 20 to 450 Hz are typically characteristic of electrical muscle activity so a reference signal of 1 kHz may be used. The signal received at a sensor is analyzed to determine the magnitude of the reference signal present. In this case the amplitude of the reference signal directly correlates to the Impedance of the skin circuit at the given frequency. This solution is less prone to false positives than the method discussed above and can measure the changes in the skin contact continuously with the smallest possible latency. However, as it relies on specialized circuitry to generate the AC signal the approach requires more expensive ADC devices which may not always be practical for a given solution.

There is a need for an improved method of determining the contact of an EMG sensor with the skin.

### SUMMARY OF THE INVENTION

There is provided a method of determining a measure of contact of an EMG sensor with the skin of a human or animal subject, the method comprising:
receiving data captured by the EMG sensor;
calculating a spectral entropy of the received data over a first time period in respect of a predetermined frequency band;
determining a measure of contact for the first time period in dependence on the spectral entropy of the received data; and
processing the received data of the first time period in dependence on the measure of contact.

Calculating the spectral entropy may comprise calculating the Shannon entropy of a power distribution of an EMG sensor signal represented in the received data.

Determining the measure of contact may comprise forming a measure of contact which is proportionate to the spectral entropy of the received data such that a high degree of spectral entropy is indicative of poor contact, and a low degree of entropy is indicative of good contact.

Calculating the spectral entropy may comprise:
determining a power spectrum of the received data;
determining a component spectral entropy for each of a plurality of frequency sub-bands of the power spectrum over the first time period, the plurality of frequency sub-bands spanning the predetermined frequency band; and
averaging the component spectral entropies over the frequency sub-bands so as to form the spectral entropy.

Determining the component spectral entropy may comprise calculating the Shannon entropy of the power distribution of the respective frequency sub-band.

Calculating the spectral entropy may further comprise clamping to zero values of spectral entropy in the frequency sub-bands where the spectral entropy is below a third threshold.

The method may further comprise providing feedback to the human or animal subject indicative of the measure of contact.

Processing the received data may comprise discarding the data of the first time period and/or marking the data of the first time period as not for use if the measure of contact is below a first threshold.

Processing the received data may comprise marking the data of the first time period as unreliable if the measure of contact is below a second threshold, the second threshold being lower than the first threshold.

The method may further comprise down-weighting the data of the first time period in subsequent processing of the data if the measure of contact is below the second threshold.

The method may further comprise providing an indication of the measure of contact to the user.

The method may further comprise repeating the method for a sequence of overlapping time windows, each time window being of the first time period.

The time windows in a sequence of time windows may overlap such that the period of overlap between adjacent time windows of the sequence is substantially greater than the time by which subsequent time windows of the sequence are offset from one another.

Each time window may be offset by 100 ms relative to adjacent time windows.

The first time period may be 5 seconds.

The received data may comprise differential EMG data obtained by a pair of EMG sensors.

The predetermined frequency band may encompass the biological signals of interest.

The predetermined frequency band may be 20 to 450 Hz.

The method may further comprise performing the method for a plurality of EMG sensors of the animal or human subject and determining an active cancellation signal by combining the signals detected by the plurality of EMG sensors in dependence on their respective measures of the degree of contact and injecting the active cancellation signal into the body.

Determining the active cancellation signal may comprise weighting the signal detected by each EMG sensor in dependence on its measure of degree of contact.

### DESCRIPTION OF THE DRAWINGS

The present invention will now be described by way of example with reference to the accompanying drawings. In the drawings:

FIG. 1 is a schematic diagram of a system configured in accordance with the principles set out herein.

FIG. 2 illustrates eyeglasses which include EMG sensors to which the methods described herein may be applied.

FIG. 3 shows an example of the spectral entropy of a data window computed for 8 frequency sub-bands.

FIG. 4 illustrates an average of the spectral entropies of the frequency sub-bands.

FIG. 5 is a flowchart illustrating a method of determining a measure of contact of EMG sensors in accordance with the principles described herein.

### DETAILED DESCRIPTION

The following description is presented to enable any person skilled in the art to make and use the invention and is provided in the context of a particular application. Various modifications to the disclosed embodiments will be readily apparent to those skilled in the art.

The general principles defined herein may be applied to other embodiments and applications without departing from the spirit and scope of the present invention. Thus, the present invention is not intended to be limited to the embodiments shown but is to be accorded the widest scope consistent with the principles and features disclosed herein.

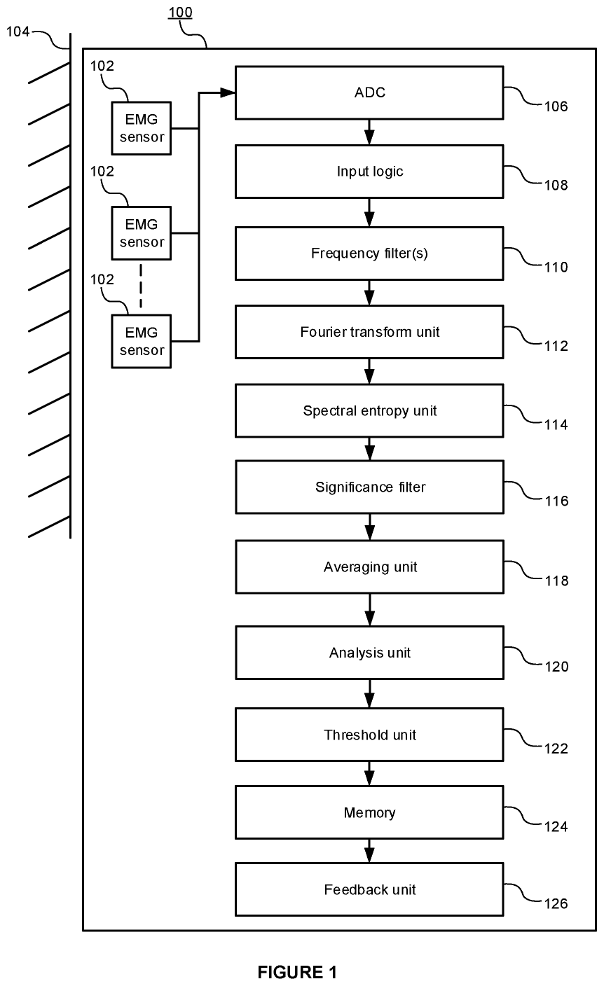

FIG. 1 shows a system 100 comprising one or more EMG sensors 102. The EMG sensors 102 are configured to measure the electric potential generated muscle activity. Hence it is desirable that EMG sensors 102 are in good contact with the skin 104 of a human or animal subject.

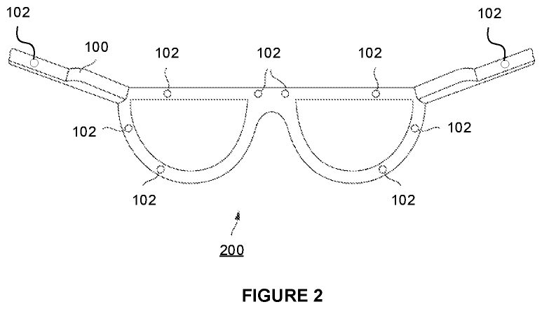

The system 100 may be incorporated into a wearable device 200, as shown in FIG. 2. The wearable device 200 may comprise glasses or a VR/AR headset (generally facewear). The EMG sensors 102 may be positioned about the device 200 in order to detect facial muscle activity. For instance, in the example shown in FIG. 2, the device 200 may comprise EMG sensors 102 positioned on the arms of glasses in order to detect the activity of the posterior auricular muscle, on the rims of the glasses to detect activity of the orbicularis oculi and the zygomaticus, and above the nosepiece to detect activity of the procerus. The sensors 102 may be configured to detect patterns facial muscle activity characteristic of one or more facial expressions. For example, activation of the zygomaticus is characteristic of a smile and activation of the procerus is characteristic of a frown.

A wearable device 200 incorporating system 100 may be used for leisure/entertainment purposes, for example to control a character's movement and/or facial expressions in a video game. The wearable device 200 may also have medical applications, for example to assist in the rehabilitation of those having issues with their facial muscles. The device 200 can be used to monitor and provide feedback to/about a patient recovering from a stroke or Bell's palsy. For each of these applications, it is important that EMG sensors 102 are in good contact with the skin in order to provide reliable information regarding the facial muscle activity of the subject.

The contact between the sensors and the skin can be significantly affected by the bunching of skin during certain facial expressions. For example, the contact between a sensor above the bridge of the nose may be significantly different during a frown than when relaxed, due to the skin bunching.

It should be apparent that system 102 is also suitable for use when monitoring the electrical activity of other muscles. For example, the system 100 can provide important information regarding the contact of sensors with a patient skin while monitoring their heart or other muscles, for medical or sports science applications.

The signals captured by measured the EMG sensors 102 are converted into digital signals by an analogue-to-digital converter (ADC) 106. The ADC 106 may store the digitised signal in a memory, for example a short-term memory such as a cache or RAM.

Input logic 108 is configured to receive the signal from the ADC 106 or short-term memory and pass on the data captured by the EMG sensors 102 in a given time window. The data captured by the EMG sensors 102 in a given time window may be referred to as a data window. For example, the input logic 108 may be configured to load 5 seconds worth of data from the short-memory or to receive 5 seconds worth of data from the ADC 106. A time window may be for example, 1, 2, 3, 4, 5, or 10 seconds in length. Hence, the subsequent processing can be performed on one or more windows of data. Preferably, where a sequence of windows is to be processed, the windows overlap. The windows in a sequence of windows may overlap such that the period of overlap between adjacent windows is substantially greater than the time by which subsequent windows are offset from one another. For example, each window may comprise 5 seconds of data and each window may be delayed by 50 ms relative to the preceding window. In other words, the input logic may be configured to divide the received data into a plurality of overlapping windows. This ensures that all relevant events (sensor disconnection, movement artifacts etc.) will fall within a plurality of windows. This adds redundancy and ensures that no event will be missed by, for example, occurring only at the very start/end of a window.

The data window may then undergo pre-processing. For example, one or more frequency filters 110 may be applied. The frequency filters 110 may comprise one or more band-stop filters. The band-stop filters may be configured to remove noise cause by mains AC power (50 Hz in the UK) and harmonics associated with the AC power supply. The frequency filters 110 may additionally or alternatively comprise one or more band-pass filters configured to remove frequencies in which biosignals will not be present. As the primary biosignals of interest in EMG readings lie in the approximate range of 20 to 450 Hz, the band-pass filters may be configured to remove frequencies below 20 Hz and/or above 450 Hz.

System 100 may further comprise a Fourier transform unit 112 configured to apply a discrete Fourier transform to convert the data window into the time-frequency domain. In other words, the Fourier transform unit 112 calculates the spectrogram of the received data window. In other examples, other types of transform could be used, such as a Laplace or Z-transform.

The system 100 may further comprise a Spectral entropy unit 114 configured to determine the spectral entropy of the data window. The spectral entropy (SE) of a signal is a measure of its spectral power distribution. The SE treats the signal's normalized power distribution in the frequency domain as a probability distribution and calculates the Shannon entropy of that probability distribution function. The Shannon entropy in this context is the spectral entropy of the signal. The inventors have appreciated that by determining the SE of an EMG signal, the degree of contact between the one or more EMG sensors 102 can be assessed, and the data captured by these sensors can be handled differently in dependence on the degree of contact. A high degree of entropy is indicative of poor contact, whereas a low degree of entropy is indicative of good contact. The measure of spectral energy can therefore be used as a measure of the degree of contact of a sensor with the human body.

For a signal V(n), the power spectrum is S(m)=|X(m)|², where X(m) is the discrete Fourier transform of V(n). The probability distribution P(m) is then:

$$P ( m ) = \frac{S ( m )}{{Σ}_{i} S ( i )} .$$

The spectral entropy H follows as:

$$H = - {\sum }_{m = 1}^{N} P ( m ) {log}_{2} P ( m )$$

where N is the total number of frequency values.

When the time-frequency power spectrum S(t, f) has been determined, the probability distribution becomes:

$$P ( m ) = \frac{S ( t , m )}{{Σ}_{f} {Σ}_{t} S ( t , f )}$$

and the spectral entropy H is still defined as above.

To compute the instantaneous spectral entropy given a time-frequency power spectrum S(t, f), the probability distribution at a time t is:

$$P ( t , m ) = \frac{S ( t , m )}{{Σ}_{f} S ( t , f )}$$

and the spectral entropy at a time t is:

$$H ( t ) = - {\sum }_{m = 1}^{N} P ( t , m ) {log}_{2} P ( t , m ) .$$

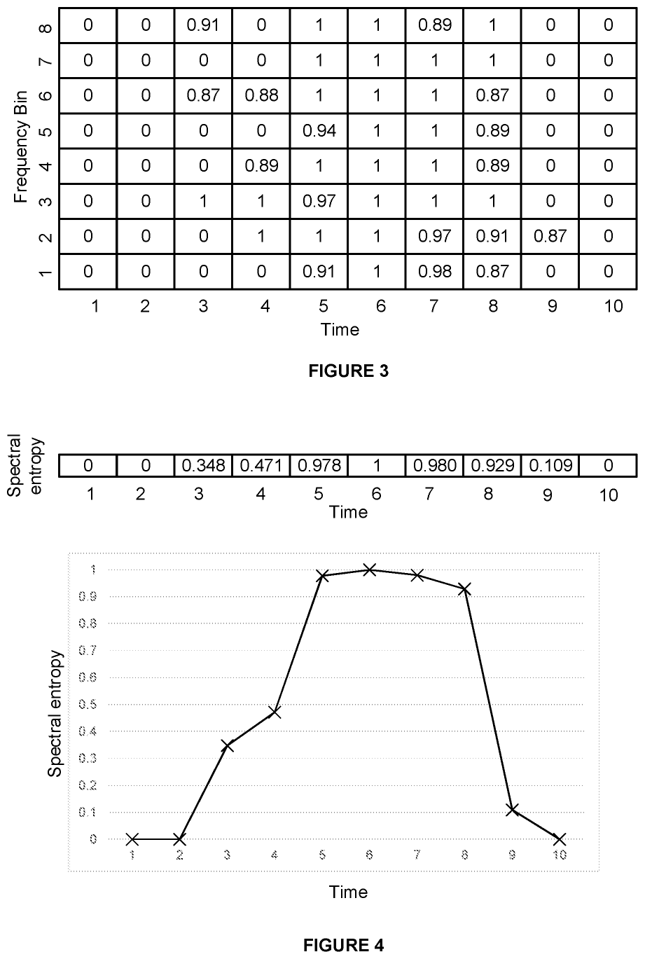

FIG. 3 shows an example of the spectral entropy of a data window computed for 8 frequency sub-bands. For example, the frequency sub-bands may be 50 Hz intervals that span the range 50 to 450 Hz.

The system 100 may comprise a further filter 116 in order to filter out weak entropies and keep significant changes in spectral entropy. This filter 116 may be referred to as a significance filter 116. The significance filter 116 may be configured to filter out weak entropies by, for example, setting all spectral entropies below a threshold to zero. For example, all spectral entropies below 0.86 may be set to zero. This helps to remove low-level variations in spectral entropy that may not be characteristic of lost/reduced sensor contact. A threshold of 0.86 has been empirically determined to provide a good balance of removing weak readings while retaining relevant values.

The significance filter 116 may additionally or alternatives comprise a filter to detect and remove outliers. For example, the significance filter 116 may apply a Hampel filter. Applying a Hampel filter comprises, for each sample, computing the median of a window comprising the sample and its six surrounding samples. The standard deviation of each sample relative to the media of the window is estimated using the median absolute deviation. If a sample differs from the median by more than three standard deviations, it is replaced with the median. Other forms of outlier removal and median filtering methods may be applied.

The spectral entropies shown in FIG. 3 have undergone significance filtering, and hence only significant, non-outlier spectral entropies are retained.

The system 100 may comprise an averaging unit 118 configured to determine the average of the spectral entropies of the frequency sub-bands for each data window. The averaging unit 118 may be configured to determine the mean or the median of the spectral entropies. In the example of FIG. 3, the mean of the 8 frequency sub-bands are determined for each data window (or time step). This averaging ensures that an anomalously high spectral entropy values for a few frequency sub-bands or a single frequency sub-band do not negatively impact the overall analysis. This also acts to reduce the amount of data. In the example of FIG. 3, the data is reduced from 8 time series to a single time series.

The averaged spectral entropies are shown in FIG. 4. It has been established that the spectral entropy may be used as a measure of the degree of contact of an EMG sensor with the body—with a high spectral entropy indicating poor contact, and a low spectral entropy indicating good contact. In FIG. 4, a characteristic loss of contact of EMG sensors 102 is shown, with the onset of the loss of contact occurring at time t=2 and resolving at time t=10.

The system may comprise an analysis unit 120 configured to determine a measure of sensor contact in dependence of the spectral entropy. In the simplest example, the spectral entropy may be directly used as the measure of contact. The measure of contact may be a binary measure, which is 1 when the spectral entropy is 0 and is 0 otherwise. The measure of contact may be proportional (or inversely proportional) to the spectral entropy. The measure of contact may further depend on other factors, for example input from other sensors, such as optical sensors (e.g. optical flow sensors) and inertial motion units (e.g. accelerometers, gyroscopes, satellite-based navigation systems).

The system 100 may further include one or more inertial motion units (not shown). Each inertial motion unit may be associated with one or more of the sensors 102 and be configured to form a measure of the movement of those one or more sensors. The movement of sensors may provide a further measure of the degree of contact between those sensors. Sensors experiencing movement may experience varying degrees of contact.

The system 100 comprises a threshold unit 122 configured to compare the measure of contact to a predetermined threshold. The predetermined threshold may be 0, such that the threshold unit 122 is configured to determine whether the measure of contact is non-zero. The predetermined threshold may be empirically determined such that it is indicative of a transition between good sensor contact and abnormal sensor contact. Further thresholds may be used in order to characterise other events, for example, total loss of contact of a sensor with the skin.

A further threshold, indicative of the onset of abnormality, may be used. For example, rather than disregarding data, data captured whose measure of contact exceeds a threshold indicative of the onset of abnormality but does not exceed the threshold indicative of loss of contact may be marked as less reliable data and/or downweighted in any future analysis. In other words, in dependence on a comparison of the measure of contact with one or more thresholds, the data may be stored and labelled/weighted, or completely discarded. This provides for a more advance data recording system that is not simply binary in its decisions. It should be apparent that multiple thresholds may be used to characterise the sensor contact and the data. For example, data may be characterised as: unusable (no contact), unreliable (abnormal contact), and reliable (full contact). In the example shown in FIG. 3 discussed above in which spectral entropies above 0.86 are retained, the threshold 0.86 represents the onset of no contact. A lower threshold (e.g. 0.5) may represent the onset of abnormal contact (i.e. spectral entropies between 0.5 and 0.86 are indicative of abnormal contact).

Suitable thresholds may be established in any suitable manner—e.g. empirically and/or through a calibration process. The predetermined threshold may be a static value or the threshold unit 122 may be configured to dynamically adjust the threshold during operation. The predetermined threshold may be set during a calibration process such that, in a given signal environment, the threshold may be set in dependence on the magnitude of the spectral entropy at the point a sensor 102 is deemed to lose contact with the body 104 (e.g. when the sensor loses physical contact with the body or when the sensor is some predefined distance from the body).

The system 100 is configured to process data captured by the one or more EMG sensors 102 in dependence on the measure of contact, and, optionally, on the one or more predetermined thresholds. For example, the system 100 may discard or mark data as unreliable from the respective sensor if the measure of contact is indicative of poor/loss of contact (e.g. the measure of contact is lower than a first predetermined threshold). Discarding the data may comprise deleting it from short-term memory and/or not storing it in long-term memory 124. The system may be configured to, in respect of data for which the measure of contact is not indicative of poor/loss of contact, store the data in long-term memory 124. In some examples, the system may be configured to process data captured by the one or more EMG sensors 102 in dependence on the measure of contact by, where the measure of contact indicates that the data is unreliable but not unusable, down-weighting the unreliable data.

The system 100 may be configured to provide feedback indicative of the measure of contact to a user via feedback unit 126. The user may be the subject to which the EMG sensors 102 are applied, or the user may be another person, for example a medical professional. The feedback unit 126 may comprise one or more of: a display, a speaker, a light, and a vibration system. Hence, the feedback may comprise one or more of: visual feedback (such as a video or one or more flashing lights), audible feedback (such as recorded or artificial speech, or one or more tones or musical sequences), and haptic feedback. The magnitude of the feedback may be proportional to the measure of contact. For example, feedback of a greater magnitude may be provided when the measure of contact is lesser. The feedback unit 126 may be configured to prompt the user and/or the subject to correct the contact of the EMG sensors 102.

The system 100 is able to detect and act on suboptimal sensor contact faster than methods that inject a reference signal into the body at a frequency different from the biological signal of interest. Comparative tests have indicated that the method applied by system 100 detects suboptimal sensor contact several (˜5) seconds before the aforementioned method. It should be appreciated that the method implemented by system 100 may combined with other methods of sensor contact evaluation in order to provide a robust and reactive measure.

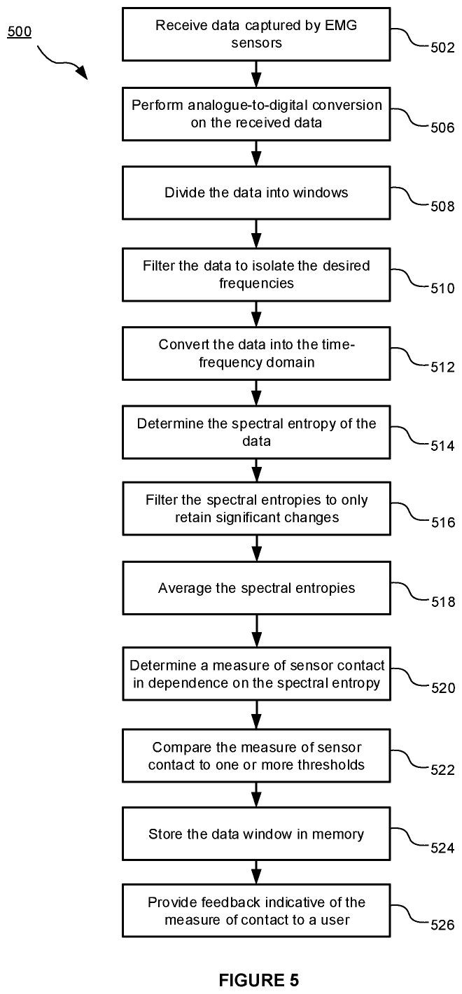

FIG. 5 shows a method 500 of determining a measure of contact of EMG sensors 102. At step 502, data captured by one or more EMG sensors 102 is captured. The captured data then undergoes analogue-to-digital conversion at step 506. The digitized data may be stored in memory, for example a short-term memory such as a cache or RAM.

At step 508, the data is divided into windows. This may be done by input logic 108 as described above. For example, the data may be divided into a plurality of overlapping windows. The windows may be, for example, 5 seconds in length. Each window may be delayed by, for example, 50 ms relative to the previous window.

At step 510, the data may be filtered in order to isolate the desired frequencies. In general, the primary biosignals of interest in EMG readings lie in the approximate range of 20 to 450 Hz. Hence, the data may be filtered to remove frequencies below 20 Hz and above 250 Hz. Furthermore, common unwanted noise frequencies may be filtered out, such as that generated by AC mains electricity as described above.

At step 512, the data is converted/transformed into the time-frequency domain. This may be performed by, for example, operating a Fourier transform on the data. The resultant data may be displayed as a spectrogram.

The spectral entropy of the data is then determined at step 516. A described above, the spectral entropy of a signal is a measure of its spectral power distribution. Step 512 may be considered a distinct step, or it may be considered to be part of step 514.

At step 516 the spectral entropies determined in the preceding step are filtered in order to only retain significant changes in spectral entropy. As described above, this may be done by setting all spectral entropies below a threshold to zero, for example all spectral entropies below 0.86 may be set to zero Additionally or alternatively, outliers may be detected and removed using a filtering method, such as applying a Hampel filter.

At step 518 the spectral entropies of the frequency sub-bands for each time step may be averaged. As described above, this may comprises determining the mean or the median of the spectral entropies. This averaging ensures that an anomalously high spectral entropy values for a few frequency sub-bands or a single frequency sub-band do not negatively impact the overall analysis. This also acts to reduce the amount of data. In the example of FIG. 3, the data is reduced from 8 time series to a single time series.

At step 520 a measure of sensor contact is determined in dependence on the spectral entropy. As described above, the measure may be a binary value or a continuous variable. The measure may further depend on other factors, for example input for one or more sensors of a different type (i.e. not EMG sensors), such as optical sensors or inertial motion units.

At step 522, the measure of contact is compared to a predetermined threshold. The predetermined threshold may be empirically determined such that it is indicative of a transition between good sensor contact and abnormal sensor contact. Further thresholds may be used in order to characterise other events, for example, total loss of contact of a sensor with the skin.

At step 524, the data window may be stored in memory. The data window may only be stored in memory if the measure of contact exceeds the predetermined threshold. In addition to the predetermined threshold described with respect to step 522, a further threshold, indicative of the onset of abnormality, may be used. For example, rather than disregarding data, data captured whose measure of contact exceeds a threshold indicative of the onset of abnormality but does not exceed the threshold indicative of total loss of contact may be marked as less reliable data and/or downweighted in any future analysis. In other words, in dependence on a comparison of the measure of contact with one or more thresholds, the data may be stored and labelled/weighted, or completely discarded.

At step 526, feedback indicative of the measure of contact may is provided to a user. The feedback may comprise one or more of: a display, a speaker, a light, and a vibration system. The magnitude of the feedback may be proportional to the measure of contact.

Applications of the present invention include any system in which the contact between biological electrical sensors and a body cannot be assured. Any use of these sensors which does not use gels and adhesives, such as personal users in a non-clinical setting can benefit from the present invention. One such application of the present invention is face monitoring headwear (and/or facewear). Sensors intended for contact with the face often suffer from a poor degree of contact with the skin due to the frequent, varied and unpredictable motion of the face. Facial movements may result from facial expressions, talking, eating, twitches and tics. Furthermore, in the face, multiple antagonist muscles insert at the same point which makes facial muscle activations more likely to cause sensor lift and/or impedance changes at sensors arranged to monitor those facial muscles. It is therefore difficult to provide sensors on facewear which are held in good contact with the skin at all times.

The particularly dynamic nature of facial movements make adhesive sensors unsuitable for use on the face. For these reasons, facewear mounted sensors can particularly benefit from compensation for the degree of contact provided by the present invention. Glasses 200 or other headwear including the system 100 are particularly suited for measuring biological electrical activity in an unobtrusive and inconspicuous manner. Glasses or other headwear including the system 100 allow the biological electrical activity to be measured without impeding a wearer's movement or vision.

The various modules and units described herein may be embodied in hardware on an integrated circuit. The system 100 described herein may be configured to perform any of the methods described herein. Generally, any of the functions, methods, techniques or components described above can be implemented in software, firmware, hardware (e.g., fixed logic or fixed function circuitry), or any combination thereof. The terms “module,” “functionality,” “component”, “element”, “unit”, “block” and “logic” may be used herein to generally represent software, firmware, hardware, or any combination thereof. In the case of a software implementation, the module, functionality, component, element, unit, block or logic represents program code that performs the specified tasks when executed on a processor. The algorithms and methods described herein could be performed by one or more processors executing code that causes the processor(s) to perform the algorithms/methods. Examples of a computer-readable storage medium include a random-access memory (RAM), read-only memory (ROM), an optical disc, flash memory, hard disk memory, and other memory devices that may use magnetic, optical, and other techniques to store instructions or other data and that can be accessed by a machine.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A method of determining a measure of contact of an EMG sensor with the skin of a human or animal subject, the method comprising:
receiving data captured by the EMG sensor;
calculating a spectral entropy of the received data over a first time period in respect of a predetermined frequency band;
determining a measure of contact for the first time period in dependence on the spectral entropy of the received data; and
processing the received data of the first time period in dependence on the measure of contact.

2. The method of claim 1, wherein calculating the spectral entropy comprises calculating the Shannon entropy of a power distribution of an EMG sensor signal represented in the received data.

3. The method of claim 1, wherein the determining the measure of contact comprises forming a measure of contact which is proportionate to the spectral entropy of the received data such that a high degree of spectral entropy is indicative of poor contact, and a low degree of entropy is indicative of good contact.

4. The method of claim 1, wherein calculating the spectral entropy comprises:
determining a power spectrum of the received data;
determining a component spectral entropy for each of a plurality of frequency sub-bands of the power spectrum over the first time period, the plurality of frequency sub-bands spanning the predetermined frequency band; and
averaging the component spectral entropies over the frequency sub-bands so as to form the spectral entropy.

5. The method of claim 4, wherein determining the component spectral entropy comprises calculating the Shannon entropy of the power distribution of the respective frequency sub-band.

6. The method of claim 4, wherein calculating the spectral entropy further comprises clamping to zero values of spectral entropy in the frequency sub-bands where the spectral entropy is below a third threshold.

7. The method of claim 1, further comprising providing feedback to the human or animal subject indicative of the measure of contact.

8. The method of claim 1, wherein the processing the received data comprises discarding the data of the first time period and/or marking the data of the first time period as not for use if the measure of contact is below a first threshold.

9. The method of claim 8, wherein the processing the received data comprises marking the data of the first time period as unreliable if the measure of contact is below a second threshold, the second threshold being lower than the first threshold.

10. The method of claim 9, further comprising down-weighting the data of the first time period in subsequent processing of the data if the measure of contact is below the second threshold.

11. The method of claim 1, further comprising repeating the method for a sequence of overlapping time windows, each time window being of the first time period.

12. The method of claim 11, wherein the time windows in a sequence of time windows may overlap such that the period of overlap between adjacent time windows of the sequence is substantially greater than the time by which subsequent time windows of the sequence are offset from one another.

13. The method of claim 1, wherein the received data comprises differential EMG data obtained by a pair of EMG sensors.

14. The method of claim 1, wherein the predetermined frequency band encompasses the biological signals of interest.

15. The method of claim 1, further comprising performing the method for a plurality of EMG sensors of the animal or human subject and determining an active cancellation signal by combining the signals detected by the plurality of EMG sensors in dependence on their respective measures of the degree of contact and injecting the active cancellation signal into the body.

16. The method of claim 15, wherein determining the active cancellation signal comprises weighting the signal detected by each EMG sensor in dependence on its measure of degree of contact.

17. The method of claim 1, wherein the EMG sensor is arranged to detect facial muscle activity of the human subject.

18. The method of claim 17, wherein the skin is the facial skin of the human subject.

19. An apparatus for determining a measure of contact of an EMG sensor with the skin of a human or animal subject, the apparatus comprising:
input logic configured to receive data captured by the EMG sensor;
a spectral entropy unit configured to calculate a spectral entropy of the received data over a first time period in respect of a predetermined frequency band; and
an analysis unit configured to determine a measure of contact for the first time period in dependence on the spectral entropy of the received data;
wherein the apparatus is configured to process the received data of the first time period in dependence on the determined measure of contact.

20. The apparatus of claim 19, wherein the apparatus is facewear and the EMG sensor is arranged such that, in use when the facewear is worn by a human subject, the EMG sensor is configured to capture EMG signals from one or more facial muscles of the human subject.
